(19) United States
(12) Patent Application Publication
(10) Pub. No.: US 2024/0390180 A1
(43) Pub. Date: Nov. 28, 2024

(54) HYPERBARIC HYPER-HYPO THERMAL THERAPY SYSTEM

(71) Applicant: Life Express LLC, San Antonio, TX (US)

(72) Inventor: Raymond Paul RENEAU, San Antonio, TX (US)

(21) Appl. No.: 18/670,473

(22) Filed: May 21, 2024

Related U.S. Application Data
(60) Provisional application No. 63/626,269, filed on Jan. 29, 2024.
(60) Provisional application No. 63/468,393, filed on May 23, 2023.

(51) Int. Cl. A61F 7/00; A61G 10/02; A61M 16/10

(52) U.S. Cl. CPC A61F 7/0053; A61G 10/026; A61F 2007/0055; A61M 16/1005

(57) ABSTRACT

The present disclosure provides a hyperbaric thermal therapy system for medical applications. The disclosed system allows for independent control of pressure, temperature, and gas composition in the breathing system supplying breathing gas to the patient, and optionally of the environment surrounding a patient in a hyperbaric chamber, which allows for a wide range of hyperbaric thermal therapy profiles. Such hyperbaric thermal therapy profiles may be utilized in combination with other treatments, including medicament-based treatments, to enhance the therapeutic outcomes of such other treatments.

### CROSS-REFERENCE TO RELATED APPLICATIONS

This application claims benefit of U.S. provisional patent application Ser. No. 63/468,393, filed May 23, 2023, and U.S. provisional patent application Ser. No. 63/626,269, filed Jan. 29, 2024, each of which is herein incorporated by reference in its entirety.

### FIELD

This disclosure relates to hyperbaric chamber systems used for medical applications, and in particular, control systems for hyperbaric chambers. The disclosed hyperbaric chamber systems allow for the independent control of pressure, temperature, and gas composition in the breathing system(s) supplying therapeutic breathing gases to a patient in a hyperbaric chamber, as well as the independent control of pressure, temperature, and gas composition in the pressurized environment surrounding the patient in the hyperbaric chamber.

### BACKGROUND

Clinical hyperbaric therapy is indicated for the treatment of various medical conditions and physical training regimens. Typically, hyperbaric therapy involves positioning a patient in a clinical hyperbaric chamber, which is then pressurized with pure oxygen, air, or other gases for treatment at pressure levels 1.5 to 3 times higher than standard atmospheric pressure. The purpose of hyperbaric therapy is to restore and/or increase the patient's blood and tissue oxygen levels to equal or greater levels than normal levels.

In many cases, thermal therapy is simultaneously applied with hyperbaric therapy. However, existing single-patient clinical hyperbaric systems rely on externally heating or cooling specific areas of a patient's body topically in order to create a thermal flow from an external source to an interior tissue. This can present numerous problems.

For example, some current clinical hyperthermal hyperbaric systems utilize heating pads that are applied topically to a patient or indwelling heating probes that are inserted invasively into a tissue of the patient. These methods typically require the application of temperatures higher than the desired therapeutic temperatures in order to create a thermal flow from the heat source to the targeted tissue(s). Yet, differential temperatures must generally be limited in order to prevent tissue damage from the higher external temperatures that are required to effectively create therapeutic temperature gradients to a target site.

Likewise, radiation heating methods can also have secondary effects, and temperatures must be limited in order to minimize or prevent damage to surrounding tissue.

Similar to hyperthermal treatments, hypothermal applications require cooling of the whole body to lower-than-normal temperatures. Conventional hypothermal treatments are typically performed by surrounding the whole body in ice or by similar thermal cooling techniques, which lack a targeted effect and are difficult to implement in a hyperbaric environment.

Therefore, there is a need in the art for hyperbaric systems with improved heating and/or cooling systems for medical and/or therapeutic objectives.

### SUMMARY

The present disclosure describes a unique bi-level therapeutic hyperbaric thermal system that facilitates heating and/or cooling of internal body temperature at the tissue level while simultaneously providing for independent external heating or cooling of the body to either resist or enhance heat flow to or from the whole body. This technology has many therapeutic advantages not otherwise achievable without invasive procedures and/or tissue damage as caused by indwelling heating probes, topical heating pads, or radiation tissue damage.

In some examples, the present disclosure provides a system for administering hyperbaric thermal therapy, the system comprising: a hyperbaric chamber; and a gas and temperature control module in communication with the hyperbaric chamber, the gas and temperature control module configured to independently control: a composition, temperature, and pressure of inhalation gases supplied to a patient in the hyperbaric chamber, and a composition and temperature control of internal atmospheric pressurization gas(es) supplied to the hyperbaric chamber.

In some examples, the present disclosure provides a system for administering hyperbaric thermal therapy, the system comprising: a gas and temperature control module configured to be removably coupled to a hyperbaric chamber, the gas and temperature control module configured to independently control: a composition, temperature, and pressure of therapeutic inhalation gases for supply to a patient in the hyperbaric chamber, and a composition and temperature control of internal atmospheric pressurization gas(es) for supply to the hyperbaric chamber.

In some examples, the present disclosure provides a system for administering hyperbaric thermal therapy, comprising: a hyperbaric chamber; a chamber base for supporting the hyperbaric chamber; and a gas and temperature control module integrated with the chamber base or the hyperbaric chamber, the gas and temperature control module configured to independently control: a composition, a temperature, and a pressure of internal atmospheric gases for supply to the hyperbaric chamber; and a composition and a temperature of inhalation gases for supply to a patient in the hyperbaric chamber.

### BRIEF DESCRIPTION OF THE DRAWINGS

So that the manner in which the above-recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIG. 1 illustrates a schematic plan view of a hyperbaric thermal therapy system, according to certain embodiments of the present disclosure configured into separate control modules: a patient control module and a chamber control module.

FIG. 2 illustrates a schematic plan view of another hyperbaric thermal therapy system configuration, according to certain embodiments of the present disclosure for a combined control module.

FIG. 3 illustrates a schematic view of a user interface that can be utilized with the hyperbaric thermal therapy systems of FIGS. 1 and 2, according to certain embodiments of the present disclosure.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

Therapeutic thermal treatment modalities, such as hyperthermal and hypothermal therapies, have applications in cancer therapy (hyperthermia) and stroke/cardiac resuscitation and rehabilitation (hypothermia). Both are emerging therapy modalities based on the controlled use of temperature to enhance and achieve beneficial therapeutic outcomes. In fact, the National Center for Biotechnology Information (NCBI) of the National Institutes of Health (NIH) has compiled numerous articles describing local, regional, and whole-body heating methods using conductive heat flow from surface/external or invasive heat sources (probes), electromagnetic energy, or ultrasound and regional perfusion to heat areas being treated for various medical conditions, including cancer.

As a further example, the National Cancer Institute (NCI) has found that therapeutic hyperthermia can be administered to damage and/or kill cancer cells, or to make cancer cells more sensitive to the effects of radiation and certain anticancer drugs. A similar finding was made by the American Cancer Society (ACS). More particularly, the ACS found that heating cancer cells to temperatures above normal (up to 113° F.) made them more susceptible to radiation and to certain chemotherapy drugs. Likewise, at the Duke University Department of Radiation Oncology, researchers found that using hyperthermia to raise body temperatures from 103° F. to 108° F. during cancer treatment obtained desired therapeutic results, as tumors were more vulnerable to radiation and chemotherapy, and further caused 30 times more drug to be delivered to tumors than with just the free drug itself.

In addition to thermal therapy, hyperbaric therapy modalities have also been found to improve therapeutic outcomes of cancer and other conditions. For example, the NCI has found that administration of oxygen under hyperbaric conditions enhances the delivery of oxygen to hypoxic tumor cells, thereby increasing their sensitivity to radiation and chemotherapy. In addition, the NCI determined that hyperbaric oxygen may improve the healing of radiation-induced injuries by improving oxygen delivery to damaged tissue.

In view of the above, embodiments of the present disclosure take advantage of the synergistic effects of hyperthermal and/or hypothermal therapy in combination with hyperbaric therapy to enhance therapeutic outcomes of various treatment modalities, including cancer treatments and other treatments for conditions such as stroke or cardiac arrest. The hyperbaric thermal therapy systems disclosed herein combine thermal therapy and hyperbaric therapy via the independent control of pressure, temperature, and gas composition of breathing gases supplied to a patient in a hyperbaric chamber as well as the gases in the environment surrounding the patient in the hyperbaric chamber.

In one uniquely synergistic aspect, the hyperbaric thermal therapy systems disclosed herein take advantage of the high thermal coefficient of helium, amplified by the pressure density factor achievable only in whole body pressurization in hyperbaric chambers, to provide loading of vital organs and tissue with high dose oxygen levels, while simultaneously producing r therapeutic hyper-hypothermal temperatures. Helium thermal conductivity is greater than any gas except hydrogen. It is also the second lightest gas, and due to the small size of its molecules, helium's diffusion rate is three times that of air. Helium is totally inert and its use in deep diving and certain medical uses are well researched and documented. While the solubility of helium is one-fourth that of nitrogen in plasma and lipid tissue, its diffusibility (speed the gas goes into and out of solution) is 2.65 times faster than nitrogen. This, combined with helium's thermal coefficient being 5.68 times greater than oxygen and amplified by the density factor in a hyperbaric chamber of two atmospheres of absolute pressure, results in the heat transfer of helium being 11.37 times greater than oxygen or air at atmospheric conditions.

Accordingly, while helium presents heating and cooling challenges in deep diving when trying to maintain normal body temperatures, helium can be beneficially utilized in clinical applications to provide rapid heating and/or cooling at the body, tissue, and/or cellular level. Using varying helium-oxygen breathing mixtures enables different thermal hyperbaric therapy profiles. For example, an 80%-20% helium-oxygen gas mixture can be utilized to rapidly affect (e.g., heat or cool) body and tissue temperatures, while a 50%-50% helium-oxygen gas mixture can be utilized to maximize tissue oxygenation at a desired therapeutic temperature and pressure.

Administering helium-oxygen breathing gases in a hyperbaric environment greatly accelerates washing out, or displacement of, tissue nitrogen and other gases in favor of helium-oxygen for accelerated thermal transfer and responsive therapeutic temperature control. The objective is an efficient method to non-invasively affect temperature and oxygen tissue levels at the cellular level. This eliminates indwelling probes and relieves tissue complications resulting from higher temperatures required for topical conductive thermal gradients.

It is known that breathing heated gas mixtures produces various physiological effects. Effects associated with helium are its low density, high diffusivity and extremely high thermal conductivity. Helium's low density reduces breathing effort and high diffusivity decreases diffusion resistance, increasing oxygenation. Helium's high heat transfer is additionally multiplied by the density factor of the increased hyperbaric pressures.

Hyperbaric thermal therapy is inherently a whole-body method. However, since there are no side effects resulting from breathing helium-oxygen at therapeutic pressures, and the high thermal conductivity of helium multiplied by the density factor makes available rapid thermal transfer, whole body hyperbaric thermal therapy greatly expands hyper-hypo thermal applications.

The hyperbaric thermal therapy systems disclosed herein may thus be utilized to administer therapeutic hyperthermia in combination with other treatments in some examples. Since one aspect of hyperthermia, in addition to direct thermal action, is increased circulation; it follows that heating the body with highly thermal conductive gas mixtures in the hyperbaric environment together with high levels of tissue oxygen would be synergistically beneficial. Potential benefits include both the synergistic therapeutic advantages noted above and a reduction in treatment time.

For example, NCBI lists a study for the ‘Feasibility of lung cancer hyperthermia using breathable perfluorochemical (PFC) liquids ‘convective hyperthermia’. The abstract states that “No method of lung hyperthermia, however, has been accepted as standard or superior”. This study involves filling one lung at a time with the heated liquid. This is an important area where hyperbaric helium-oxygen hyperthermia would be far less complicated, together with offering enhanced capabilities.

In some examples, the hyperbaric thermal therapy systems disclosed herein may be utilized to administer therapeutic hypothermia in combination with other treatments, such as medicament-based treatments. The lowering of body temperature is also experiencing growing interest and research for acute stroke and patient resuscitation after cardiac arrest, as well as an adjunctive to certain surgeries. Lowering body temperature by administrating chilled He/O2 respiratory gases to a patient in a hyperbaric chamber may have advantages over topically cooling the body with ice, or circulating a patient's blood through external cooling devices. For example, the American Heart Associate (AHA) has found that induced hypothermia is one of the most promising neuroprotective therapies, but technological limitations and homeostatic mechanisms that maintain core body temperature have impeded the clinical use of hypothermia. Further, in a research article authored by researchers at the University of Pennsylvania regarding patients resuscitated after cardiac arrest, it was suggested that “something as simple as lowering body temperature . . . could ultimately save as many as 100,000 lives a year.”

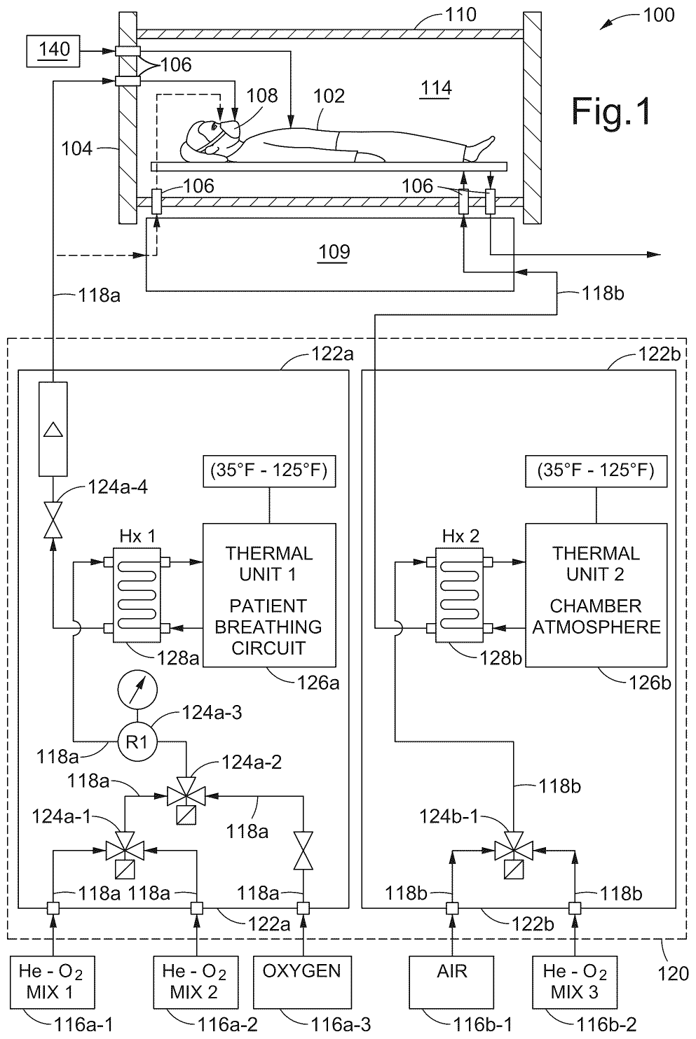

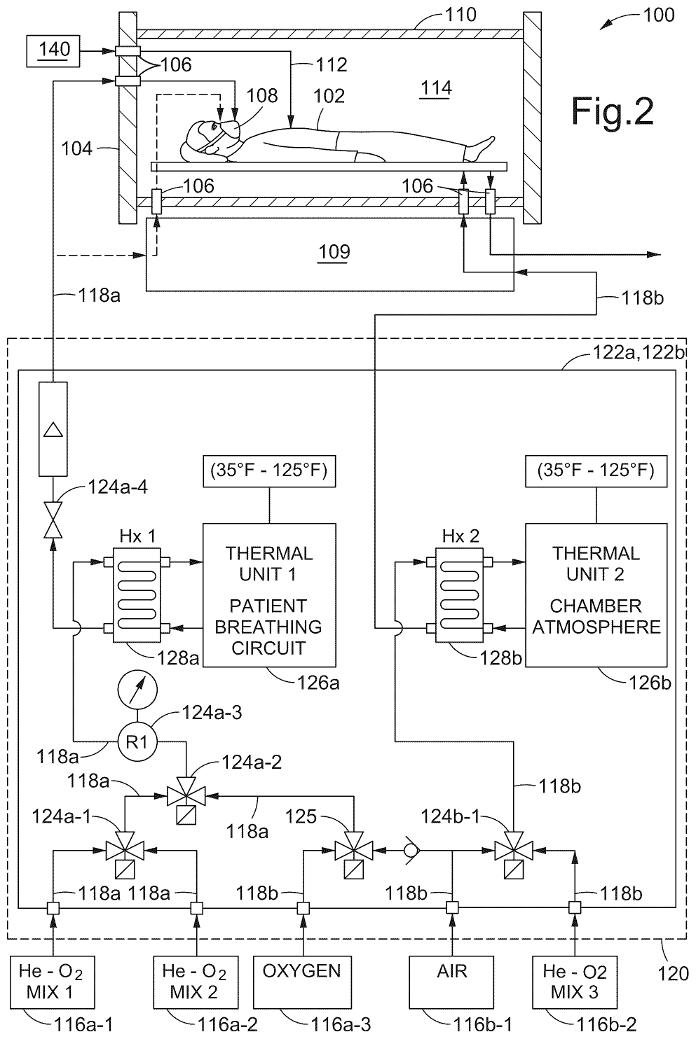

FIGS. 1 and 2 depict schematic plan views of a hyperbaric thermal therapy system 100 (sometimes referred to herein as “system 100”) for facilitating administration of hyperbaric thermal therapy to a patient 102, which may be administered simultaneously or sequentially with one or more other types of treatments, including chemical cancer therapies and treatments for conditions such as stroke or cardiac arrest, to enhance the therapeutic outcomes of such other types of treatments. The hyperbaric thermal therapy system 100 takes advantage of the synergistic effects provided by the simultaneous or sequential combination of hyperthermia and/or hypothermia, with pressure physiology and hyperbaric oxygen therapy, to facilitate or improve the therapeutic outcomes of other types of treatments. For example, whole body pressurization greatly accelerates tissue gas exchange, and enables the washing out of tissue nitrogen in exchange for oxygen and/or other inert gases inhaled at pressures greater than atmospheric.

The hyperbaric thermal therapy system 100 includes a hyperbaric thermal chamber 110 operably coupled to a gas and temperature control module 120. In certain embodiments, the gas and temperature control module 120 is disposed within, or operably coupled to, a chamber base 109, which acts as a support assembly for the hyperbaric thermal chamber 110 and/or the gas and temperature control module 120. In such examples, the gas and temperature control module 120 and/or the chamber base 109 may be physically integrated with, or built into, the hyperbaric thermal chamber 110. In certain examples, the gas and temperature control module 120 is a free-standing and separable unit that is removably coupled to the hyperbaric thermal chamber 110.

The hyperbaric thermal chamber 110 may include any suitable type of hyperbaric thermal chamber 110 in which the patient 102 can be positioned (e.g., sitting or lying) and exposed to hyperbaric conditions during therapy. For example, the hyperbaric thermal chamber 110 may include an oxygen flow-through hyperbaric chamber, an oxygen pressurized hyperbaric chamber, an air pressurized hyperbaric chamber, or other similar hyperbaric chambers. In certain embodiments, the hyperbaric thermal chamber 110 comprises a monoplace hyperbaric chamber or a multiplace hyperbaric chamber. In certain embodiments, the hyperbaric thermal chamber 110 is configured to support and operate at an internal pressure (pressure within an internal environment 114 of the hyperbaric thermal chamber 110) ranging between 1.0 to 3.0 atmospheres absolute (ATA), or 14.7 to 44.1 pounds per square inch (PSIA). In certain embodiments, the hyperbaric thermal chamber 110 is configured to support and operate at an internal temperature (temperature within the internal environment 114 of the hyperbaric thermal chamber 110) ranging between 35° F. and 125° F. (1.7° C.-51.7° C.).

In certain embodiments, the hyperbaric thermal chamber 110 includes an interface plate 104 having one or more ports 106 through which one or more cables and/or lines (e.g., supply lines 118a and/or 118b, discussed below) may be sealingly disposed for supplying the patient 102 with therapeutic inhalation gases, fluids or the like during treatment, as well as for providing power and/or electrical signals to one or more sensors and/or other electronics within the hyperbaric thermal chamber 110. For example, in certain embodiments, the ports 106 may facilitate the fluidic coupling of a breathing mask 108, which may be placed onto the patient 102 within the hyperbaric thermal chamber 110 during treatment to administer thermally controlled breathing gases thereto, with one or more gas sources 116a (116a-1-116a-3 are shown) disposed in an environment external the hyperbaric thermal chamber 110. In certain examples, the ports 106 may also facilitate the extension of one or more intravenous (IV) or infusion lines 112, which may be coupled to one or more medicament sources 140 external to the hyperbaric thermal chamber 110, into the hyperbaric thermal chamber 110 for the delivery/administration of one or more fluid-based supplemental or adjunctive therapies, including cancer chemotherapies and the like, to the patient 102 within the hyperbaric thermal chamber 110. Generally, the gas source(s) 116a may include high pressure gas cylinders, compressors, hospital/medical gas systems, combinations thereof, and the like.

The gas and temperature control module 120 enables regulation of the gases supplied to the patient 102 and/or the internal environment 114 of the hyperbaric thermal chamber 110 during treatment. In certain embodiments, the gas and temperature control module 120 facilitates control of at least the following parameters, independently: 1) a composition of breathing gas administered to the patient; 2) a temperature of the breathing gas administered to the patient; 3) a composition of the atmosphere within the hyperbaric thermal chamber 110; and/or 4) a temperature of the atmosphere within the hyperbaric thermal chamber 110. The flexibility to adjust each of the above parameters, individually, allows the provision of the most optimal hyperbaric thermal environment for any given medicament-based or other treatment to enhance the therapeutic outcomes thereof.

In certain embodiments, the gas and temperature control module 120 includes a first control circuit 122a for regulating the supply of breathing gas(es) to the patient 102 (e.g., via the breathing mask 108). For example, the first control circuit 122a may include, or be in fluid communication with, a first set of gas sources 116a (three are shown in FIGS. 1 and 2) fluidically coupled to a plurality of supply lines 118a , a first plurality of valves and/or regulators 124a (124a-1-124a-4 are shown) operably coupled to the supply lines 118a for regulating the flow of breathing gases from the gas sources 116a and through the supply lines 118a, and a first thermal unit 126a having a heat exchanger 128a operably coupled to the supply lines 118a for modulating the temperature of the breathing gases flowing through the supply lines 118a . In certain embodiments, the supply lines 118a fluidically coupling the gas sources 116a and the, e.g., breathing mask 108, may run external to the chamber base 109. In certain embodiments, the supply lines 118a may be integrated with, or disposed through, the chamber base 109.

During operation, one or more of the gas sources 116a may supply oxygen or a mixture of oxygen and helium (and other gases in certain examples), with varying concentrations to the supply lines 118a . The composition of the breathing gases flowed through the supply lines 118a , and ultimately, to the patient 102, are then actively and automatically controlled by the gas and temperature control module 120 via the plurality of valves and/or regulators 124a to achieve a desired breathing gas composition, which may be specific to one or more given therapy profiles administered to the patient 102. The plurality of valves and/or regulators 124a are further utilized to control the flow of breathing gases to the heat exchanger 128a , which can adjustably control the temperature of the breathing gases supplied to the patient 102. The heat exchanger 128a may include any suitable type of heat exchanger, such as a plate heat exchanger, double tube heat exchanger, shell and tube heat exchanger, tube in tube head exchanger, and the like. Generally, the heat exchanger 128a may adjust the temperature of the breathing gases flowed through the supply lines 118a between a range of 35° F. and 125° F.

In particular embodiments, as shown in FIG. 1, the first control circuit 122a includes or is fluidically coupled to three gas sources 116a -1, 116a -2, and 116a -3. In such embodiments, the gas sources 116a -1 and 116a -2 may supply helium-oxygen mixtures with different compositions of helium and/or oxygen, and the gas source 116a -3 may supply oxygen. During use, a first multi-port (e.g., three-way) valve 124a -1 may be utilized to mix and control the amount of the helium-oxygen mixtures supplied to the supply lines 118a from the gas sources 116a -1 and 116a -2. Meanwhile, a second valve 124a -2 may be used to mix the resulting helium-oxygen mixture from the first valve 124a -1 with oxygen supplied by the gas source 116a -3. Altogether, the valves 124a -1 and 124a -2 may control the ratio(s) of gases supplied from the gas sources 116a -1, 116a -2, and 116a -3 to control the overall composition of breathing gases supplied to the patient 102.

As noted above, the hyperbaric thermal therapy system 100 takes advantage of the high thermal coefficient and diffusion rate of the helium in the supplied breathing gases, which can be amplified by whole body pressurization in the hyperbaric thermal chamber 110, to provide direct loading of vital organs and tissues with high dose oxygen levels, while simultaneously and efficiently providing intercellular therapeutic hyper-and/or hypo thermal temperatures to improve therapeutic results.

By enabling the modulation of the supply and temperature of various helium- oxygen breathing mixtures to the patient 102, a wider range of hyperbaric thermal therapy profiles may be efficiently administered to the patient 102 in addition to the modulation of the internal atmospheric gases within the hyperbaric thermal chamber 110. The application and complications of topical conductive thermal gradients, and/or the utilization of indwelling probes are thus eliminated. For example, the thermal modulation of breathing gases supplied to the patient 102, in addition to the thermal modulation of internal atmospheric gases within the hyperbaric thermal chamber 110, allows highly efficient and rapid heating or cooling at the body core and tissue level. As a result, the need for the application of topical conductive thermal gradients can be eliminated, and localized tissue complications resulting from indwelling probes and external direct contact heating elements requiring higher temperatures to effect a thermal gradient for heating internal tissue and organs can be relieved. In certain embodiments, the efficient and rapid heating or cooling enables efficient whole body heating or cooling. In certain embodiments, the efficient and rapid heating or cooling facilitates improved heating or cooling of a targeted site or tissue of the patient's body.

In certain embodiments, the gas and temperature control module 120 further includes a second control circuit 122b for regulating the temperature and composition of the supply of internal atmospheric gas(es) to the hyperbaric thermal chamber 110. For example, the second control circuit 122b may include, or be in fluid communication with, a second set of gas sources 116b (two gas sources 116b -1 and 116b -2 are shown in FIGS. 1 and 2) fluidically coupled to a plurality of supply lines 118b , a second plurality of valves and/or regulators 124b (a single valve 124b -1 is shown) operably coupled to the supply lines 118b for regulating the flow of the chamber internal atmospheric gases from the gas sources 116b and through the supply lines 118b and a second thermal unit 126b having a heat exchanger 128b operably coupled to the supply lines 118b for modulating the temperature of the chamber internal atmospheric gases flowing through the supply lines 118b . In certain embodiments, the supply lines 118b fluidically coupling the gas sources 116b and the internal atmosphere of the hyperbaric thermal chamber 110 may run external to the chamber base 109. In certain embodiments, the supply lines 118b may be integrated with, or disposed through, the chamber base 109.

During operation, one or more of the gas sources 116b may supply oxygen or a mixture of oxygen and helium (and other gases, such as therapeutic gases, in certain examples), with varying concentrations to the supply lines 118b . The composition of the atmospheric gases flowed through the supply lines 118b and to the internal environment 114 of the hyperbaric thermal chamber 110 are then actively and automatically controlled by the gas and temperature control module 120 via the plurality of valves and/or regulators 124b to achieve a desired atmospheric gas composition in the internal environment 114, which may be specific to one or more given therapy protocols or profiles administered to the patient 102. The plurality of valves and/or regulators 124b are further utilized to control the flow of atmospheric gases to the heat exchanger 128b , which can adjustably control the temperature of the atmospheric gases supplied to the internal environment 114. In certain embodiments, the heat exchanger 128b may be substantially similar to the heat exchanger 128a , and may adjust the temperature of the atmospheric gases flowed through the supply lines 118b between a range of 35° F. and 125° F.

In particular embodiments, as shown in FIG. 1, the second control circuit 122b includes or is fluidically coupled to two gas sources 116b -1 and 116b -2. In such embodiments, the gas source 116b -1 may supply air or a similar gas mixture, and the gas source 116b -2 may supply a helium-oxygen mixture. During use, the multiport (e.g., three-way) valve 124b -1 may be utilized to mix and control the amount of the air and helium-oxygen mixture supplied to the supply lines 118b from the gas sources 116b -1 and 116b -2, respectively. Accordingly, the valve 124b -1 may control the ratio(s) of gases supplied from the gas sources 116b -1 and 116b -2 to control the overall composition of gases supplied to the internal environment 114 of the hyperbaric thermal chamber 110.

Generally, the second control circuit 122b may be operated simultaneously with the first control circuit 122a to simultaneously modulate both the breathing gases administered to the patient 102 and the atmospheric gases supplied to the hyperbaric thermal chamber 110. The simultaneous modulation of both breathing gases and atmospheric gases, in both composition and temperature, enables the provision of the most optimal hyperbaric thermal therapy profiles for a given medicament-based or other treatment being administered to the patient 102 to enhance the therapeutic outcomes thereof. In certain embodiments, the second control circuit 122b is completely independent, or separate, from the first control circuit 122a , as shown in FIG. 1. In such embodiments, each of the first control circuit 122a and the second control circuit 122b may be in stand-alone configurations, and comprise separate and corresponding gas sources 116a and 116b .

In certain other embodiments, however, the second control circuit 122b is operably coupled, or combined, with the first control circuit 122a , as shown in FIG. 2. In such embodiments, one or more of the gas sources 116a and/or 116b may supply gases to both the first control circuit 122a and the second control circuit 122b , as controlled by one or more valves and/or regulators 125. For example, in one particular embodiment, as shown in FIG. 2, the multiport (three-way) valve 125 is in fluid communication with the gas source 116a -3, which supplies oxygen, and the gas source 116b -1, which supplies air. In this embodiment, the valve 125 may be utilized to mix air from the gas source 116b -1 with the oxygen supplied to the patient 102 by the first control circuit 122a as breathing gases. Further, the air from the gas source 116b -1 may also be mixed with the helium-oxygen mixture from the gas source 116b -1 at the valve 124b -1 to form a desired composition of internal atmospheric gases for the hyperbaric thermal chamber 110.

Note that the depicted embodiments in FIGS. 1 and 2 are only exemplary, and that other arrangements of gas sources, valves, regulators, etc. for the control circuits 112a and/or 122b are further contemplated.

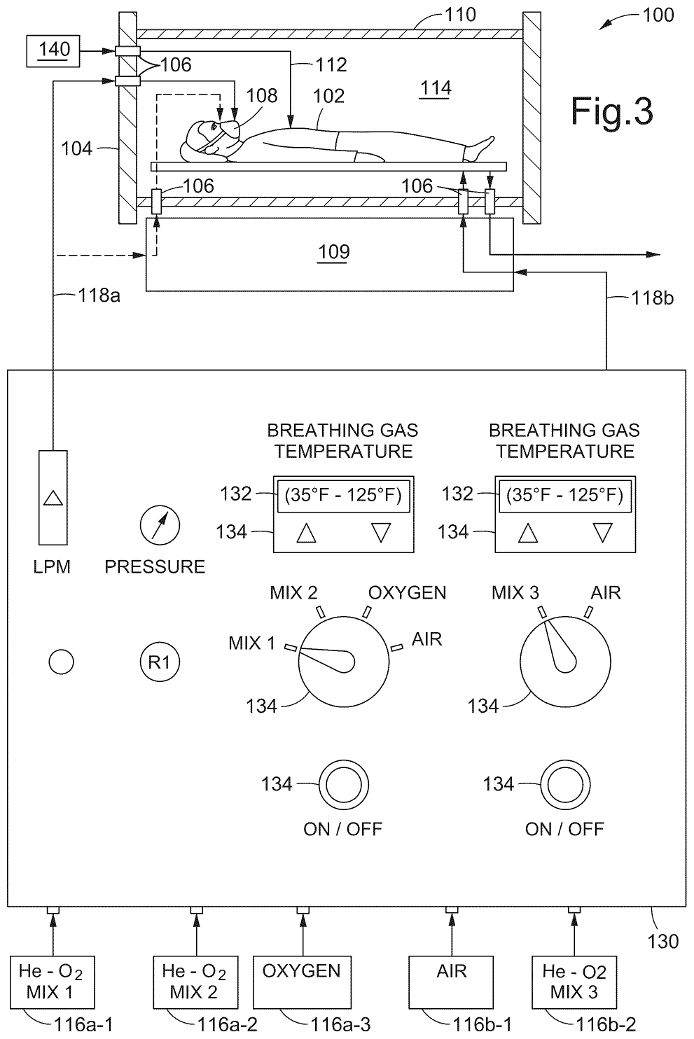

As shown in FIG. 3, the gas and temperature control module 120 may further include a dashboard 130 (e.g., a user interface) to facilitate user control of the various features of the gas and temperature control module 120. The dashboard 130 may generally include one or more display screens 132 for displaying one or more actual or selected parameters of the gas and temperature control module 120 (e.g., temperature or pressure) and one or more buttons or knobs 134 for adjusting such parameters or activating a control circuit 122 and/or various features thereof. In certain embodiments, the dashboard 130 includes one or more analog features, such as analog buttons or knobs 134. In certain embodiments, the dashboard 130 includes one or more digital features, such as digital buttons or knobs 134. In certain embodiments, the dashboard 130 includes a combination of analog and digital features.

In summary, the embodiments of the hyperbaric thermal therapy system 100 disclosed herein are configured to enable independent control of pressure, temperature, and gas composition in the internal chamber environment surrounding a patient in a hyperbaric chamber, as well as that of a breathing system supplying breathing gas to the patient. The independent control of such features facilitates a wide range of hyperbaric thermal therapy profiles which may be synergistically combined with a medicament-based or other treatment, such as a cancer or stroke treatment regimen, being administered to a patient to enhance the therapeutic outcome(s) of such other treatment.

The hyperbaric thermal therapy system 100 further eliminates direct tissue contact heating or cooling, invasive procedures, or radiation complications, while delivering a uniquely superior method for heating or cooling the body at the tissue level utilizing pressure physiology. Moreover, and importantly, indwelling, topical, or radiation heating is limited to the accuracy of the procedure to effectively cover the target area. Whereas, alternatively, the hyperbaric thermal therapy system 100 inherently provides whole body coverage that (1) ensures treatment temperatures at the targeted site and surrounding tissue are delivered, and (2) eliminates the need for differential thermal gradients with temperatures higher than required for desired therapeutic outcomes.

With the hyperbaric thermal therapy system 100, there is no tissue damage, no invasive procedures, and no exposure to radiation or risk of skin damage due to excessive thermal heating or cooling. As described herein, the utilization of an inert gas with a high thermal coefficient amplified by the density factor in the inhalation breathing gases delivered to a patient at hyperbaric pressures greater than atmospheric creates the desired therapeutic temperatures at a desired target site, as well as surrounding tissue, without any harmful or negative physiological effects.

While the foregoing is directed to embodiments of the present disclosure, other and further embodiments of the disclosure may be devised without departing from the basic scope thereof, and the scope thereof is determined by the claims that follow.

What is claimed is:

1. A system for administering hyperbaric thermal therapy, comprising:
a hyperbaric chamber; and
a gas and temperature control module in communication with the hyperbaric chamber, the gas and temperature control module configured to independently control:
a composition, a temperature, and a pressure of internal atmospheric gases for supply to the hyperbaric chamber; and
a composition and a temperature of inhalation gases for supply to a patient in the hyperbaric chamber.

2. The system of claim 1, wherein the gas and temperature control module comprises:
a first control circuit to control the composition, the temperature, and the pressure of the internal atmospheric gases for supply to the hyperbaric chamber; and
a second control circuit to control the composition and the temperature of the inhalation gases for supply to the patient in the hyperbaric chamber.

3. The system of claim 2, wherein the first control circuit and the second control circuit operate independently of each other.

4. The system of claim 3, wherein the first control circuit is in fluid communication with a first set of gas sources for supplying the internal atmospheric gases, and the second control circuit is in fluid communication with a second set of gas sources for supplying the inhalation gases.

5. The system of claim 4, wherein the first set of gas sources comprises at least one of a source of oxygen or a source of a mixture of oxygen and helium.

6. The system of claim 5, wherein the second set of gas sources comprises at least one of a source of oxygen or a source of a mixture of oxygen and helium.

7. The system of claim 3, wherein the first control circuit comprises a first thermal unit for modulating the temperature of the internal atmospheric gases for supply to the hyperbaric chamber, and the second control circuit comprises a second thermal unit for modulating the temperature of the inhalation gases for supply to the patient in the hyperbaric chamber.

8. The system of claim 7, wherein the first thermal unit is configured to modulate the temperature of the internal atmospheric gases between 35° F. and 125° F.

9. The system of claim 7, wherein the second thermal unit is configured to modulate the temperature of the inhalation gases between 35° F. and 125° F.

10. The system of claim 1, wherein the gas and temperature control module comprises a user interface to facilitate control of the gas and temperature control module, the user interface comprising at least one of a display screen, a digital button or knob, or an analog button or knob.

11. The system of claim 1, wherein the gas and temperature control module is integrated with the hyperbaric chamber.

12. The system of claim 1, wherein the gas and temperature control module is removably coupled to the hyperbaric chamber.

13. A system for administering hyperbaric thermal therapy, comprising:
a gas and temperature control module configured to be removably coupled to a hyperbaric chamber, the gas and temperature control module configured to independently control:
a composition, temperature, and pressure of internal atmospheric gases for supply to the hyperbaric chamber; and
a composition and temperature of inhalation gases for supply to a patient in the hyperbaric chamber.

14. The system of claim 13, wherein the gas and temperature control module comprises:
a first control circuit to control the composition, the temperature, and the pressure of the internal atmospheric gases for supply to the hyperbaric chamber; and
a second control circuit to control the composition and the temperature of the inhalation gases for supply to the patient in the hyperbaric chamber.

15. The system of claim 14, wherein the first control circuit and the second control circuit operate independently of each other.

16. The system of claim 15, wherein the first control circuit is in fluid communication with a first set of gas sources for supplying the internal atmospheric gases, and the second control circuit is in fluid communication with a second set of gas sources for supplying the inhalation gases.

17. The system of claim 15, wherein the first control circuit comprises a first thermal unit for modulating the temperature of the internal atmospheric gases for supply to the hyperbaric chamber, and the second control circuit comprises a second thermal unit for modulating the temperature of the inhalation gases for supply to the patient in the hyperbaric chamber.

18. The system of claim 14, wherein the gas and temperature control module comprises a user interface to facilitate control of the gas and temperature control module, the user interface comprising at least one of a display screen, a digital button or knob, or an analog button or knob.

19. A system for administering hyperbaric thermal therapy, comprising:
a hyperbaric chamber;
a chamber base for supporting the hyperbaric chamber; and
a gas and temperature control module integrated with the chamber base or the hyperbaric chamber, the gas and temperature control module configured to independently control:
a composition, a temperature, and a pressure of internal atmospheric gases for supply to the hyperbaric chamber; and
a composition and a temperature of inhalation gases for supply to a patient in the hyperbaric chamber.

20. The system of claim 19, wherein the gas and temperature control module comprises:
a first control circuit to control the composition, the temperature, and the pressure of the internal atmospheric gases for supply to the hyperbaric chamber; and
a second control circuit to control the composition and the temperature of the inhalation gases for supply to the patient in the hyperbaric chamber.
